(19) United States
(12) Patent Application Publication
(10) Pub. No.: US 2010/0190168 A1
(43) Pub. Date: Jul. 29, 2010

(54) COMPETITOR MOLECULES USEFUL FOR LOWERING NONSPECIFIC ADSORPTION OF DYE LABELED NUCLEOTIDES

(75) Inventors: Philip Buzby, Brockton, MA (US); Jayson Bowers, Cambridge, MA (US)

Correspondence Address: LANDO & ANASTASI, LLP, ONE MAIN STREET, SUITE 1100, CAMBRIDGE, MA, 02142, US

(73) Assignee: HELICOS BIOSCIENCES CORPORATION, Cambridge, MA (US)

(21) Appl. No.: 12/629,174

(22) Filed: Dec. 2, 2009

Related U.S. Application Data
(60) Provisional application No. 61/119,197, filed on Dec. 2, 2008.

(51) Int. Cl. C12Q 1/68

(52) U.S. Cl. 435/6

(57) ABSTRACT

Methods are described which enable higher signal/noise when performing surface measurements at the single molecule level. Methods are particularly useful in the field of molecular biology when performing single molecule nucleic acid sequencing by synthesis using dye labeled nucleotides. The method employs using a competitor molecule which blocks nonspecific binding of analog molecules to the surface.

### CROSS-REFERENCE TO RELATED APPLICATIONS

This application claims priority to U.S. Patent Application Ser. No. 61/119,197, filed on Dec. 2, 2008, under 35 U.S.C. §119, the contents of which are hereby incorporated by reference in its entirety.

### BRIEF DESCRIPTION OF THE FIGURES

FIG. 1 depicts the effect of competitor on Yield in a graph of megabase output/channel vs. Template density which shows that inclusion of DFMBP increases output approx. 30%.

FIG. 2 is a graph of Strand Growth Rate vs. template density which shows that inclusion of DFMBP in the sequencing by synthesis reaction has no apparent impact on growth rate.

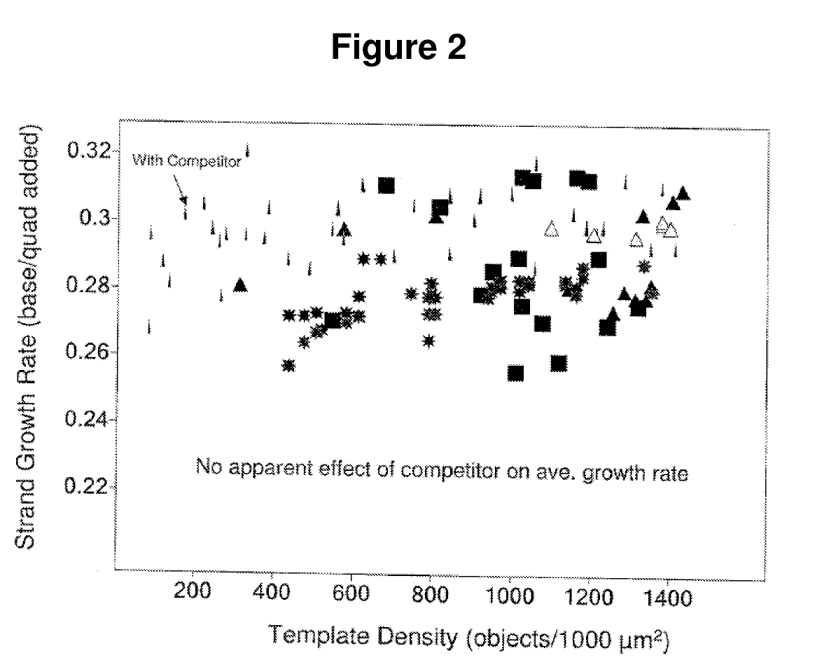

FIG. 3 is a graph of Ave. Error Rate vs. Template Density which shows that inclusion of DFMBP results in lowering the determined average error rate on the order of at least 0.5-1% with the benefit diminishing at very high template densities (density effect dominates over rinse).

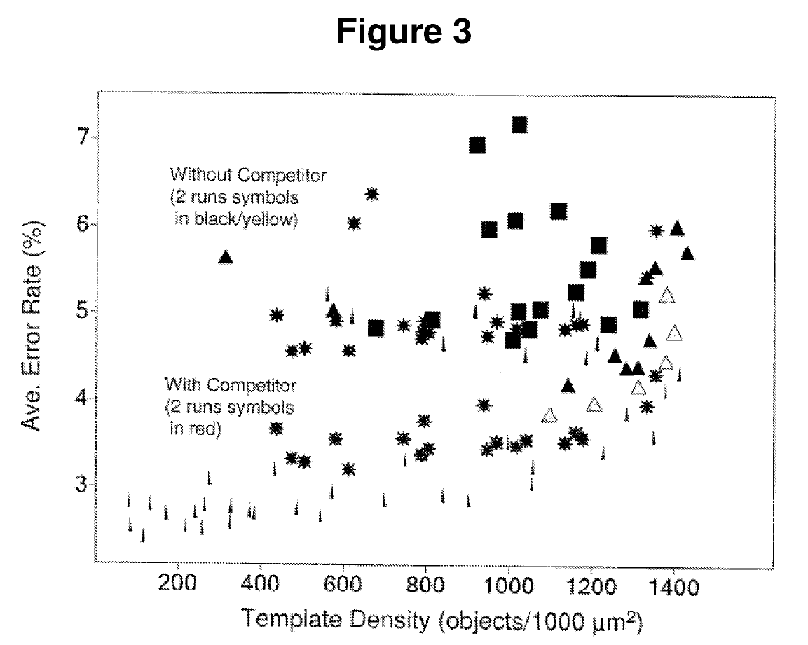

### DESCRIPTION

When performing any type of analysis on a single molecule level, one of the main issues with the measurement is to determine real signal from noise. Real signal is the result of some specific interaction, generally as a result of a biological process. Noise can be defined as any signal resulting from nonspecific adsorption of a molecule, for example to a surface, not dependent upon the biological process of interest. At the single molecule level, the signature that is measured is unable differentiate between real signal and noise. Therefore there is a need for methods which minimize the potential for noise in single molecule processes.

For example, sequencing of nucleic acids has been demonstrated at the single molecule level by several groups. Generally, dye labeled nucleotide analogs are permitted to incubate with a polymerase and other cofactors necessary for biological activity in the presence of a primer-template to form a biologically active complex. Generally there is a surface involved in which at least one member of the complex is anchored to the surface. The anchoring may be direct covalent attachment of the primer, template, or polymerase either individually or in combination. Alternatively, the anchoring may be through use of binding pairs, such as biotin:streptavidin, and one member of the binding pair is covalently attached to the surface and the other member of the binding pair is attached to any of the primer, template, or polymerase either individually or in combination.

When incubating dye labeled nucleotide(s), if there is a base pair formed between a dye-nucleotide with the next base in the template, the polymerase incorporates the dye-nucleotide onto the 3′-end of the primer. If as in the example, the dye can be imaged by TIRF to detect an incorporation event. There may be dye-nucleotides that adsorb to the surface or even the complex which are not base paired with the next base in the template and result in a noise signal when imaged.

One method published that was employed to overcome noise was the use of a method call FRET (fluorescence resonance energy transfer). FRET involves the use of 2 dyes: donor dye and an acceptor dye. FRET is very sensitive to distance between the 2 dyes, decreasing as about 1/r⁶ where r is distance. True signal is thus obtained from the FRET method with a minimum of noise since the probability of 2 dye molecules randomly occupying space close enough to FRET is exceeding small. FRET has its limitations in that the donor dye may photo bleach and thus no additional FRET signal can be generated. Additionally, in nucleic acid sequencing, if the donor dye is attached to the primer and the primer extends by base additions, as the strand grows in length and by approximately 15-20 bases the distance “r” becomes significant so as to lower the FRET efficiency. The remedy for both of these examples would be to add a new donor dye. Alternatively, if methods were known to lower the background noise to acceptable levels one would not require the use of FRET.

Another solution to reduce noise is to utilize another class of molecules or compounds which competitively block the nonspecific sticking to the surface, e.g. a “competitor”. The ideal competitor would mimic one or more structural features of the molecule producing the signal, for example, dye nucleotide analogs commonly utilized for sequencing by synthesis comprise the base, (deoxy)ribose, phosphates and dye(s). The dyes may be charged, negatively, positively or sometimes even neutral. The core of the dye is generally aromatic and thus hydrophobic in nature. Likewise, the substrate may carry a charge: positive, negative or neutral. Dye nucleotides are highly negatively charged due to the triphosphate moiety and in some cases sulfates on the dye. A competitor which mimics the negative charge and/or the polyphosphate might reduce noise. Alternatively, dyes are somewhat hydrophobic therefore inclusion of organic molecules, e.g. methanol, ethanol, acetonitrile, dimethylformamide, dimethylsulfoxide, etc. in the reaction mixture might also reduce noise.

Ideally the competitor has little or no impact on the biological reaction of interest that generates the signal. By way of example, polymerase incorporation of nucleotides liberates inorganic pyrophosphate. Typically sequencing by synthesis reactions include an enzyme to degrade pyrophosphate, e.g. a pyrophosphatase. Should the pyrophosphatase be omitted, inactive, or levels of pyrophosphate accumulate, then it is possible for the polymerase to catalyze the reverse reaction, e.g. pyrophosphorolysis, which removes or exchanges bases on the 3′-end of the primer. One would therefore not want to use inorganic pyrophosphate, supplied as various salt forms of P₂O₇⁻³, as a competitor. Single molecule reactions are extremely sensitive to trace contaminants. Another possible option for competitor is inorganic phosphate however it has been shown that within solutions of monophosphate, e.g. various salt forms of PO₄⁻³, solutions there is an equilibrium reaction which produces levels of pyrophosphate significant enough to stimulate pyrophosphorolysis.

The competitor may then be required to function differently depending upon the composition of the detectable molecule and the substrate employed. Additionally, the competitor may be used in several different ways: a substrate pre-treatment, a substrate post-treatment, a real-time treatment, and/or post real-time treatment. Examples of each are described below:

a. Pre-treatment: glass slides are washed minimally in detergent, water, competitor then washed and dried. Glass so treated is used to deposit an epoxide coating. Amine modified oligonucleotides are attached to the substrate via amine-epoxide chemistry;

b. Post-treatment: glass slides are washed minimally in detergent, water, and dried. Glass so treated is used to deposit an epoxide coating. Amine modified oligonucleotides are attached to the substrate via amine-epoxide chemistry. Substrates are washed then incubated in a blocking solution containing competitor to passivate the substrate;

c. Real-time treatment: competitor is included in the mixture of polymerase and dye nucleotide during exposure to the substrate which has attached a primer:template;

d. Post real-time treatment: competitor is included in a wash solution or imaging solution following removal of the polymerase/dye nucleotide mixture; and

e. any combination of a-d.

If the surface contains an epoxide and the competitor is totally free of reactive amines, the competitor may additionally be included with the amino-oligonucleotide during attachment to the substrate.

An example of a single molecule sequencing process follows. Epoxide-coated glass slides are prepared for oligo attachment. Epoxide functionalized 40 mm diameter #1.5 glass cover slips (slides) are obtained from Erie Scientific (Salem, N.H.). The slides are preconditioned by soaking in 3×SSC for 15 minutes at 37° C. Next, a 500-pM aliquot of 5′ aminated capture oligonucleotide (oligo dT(50)) is incubated with each slide for 30 minutes at room temperature in a volume of 80 ml. The slides are then treated with phosphate (1 M) for 4-20 hours at room temperature in order to passivate the surface. Optionally, competitor may be included in the phosphate or used in place of the phosphate for surface blocking, generally concentrations>0.1 M are desirable. Slides are then stored in 20 mM Tris, 100 mM NaCl, 0.001% Triton® X-100, pH 8.0 at 4° C. until they are used for sequencing.

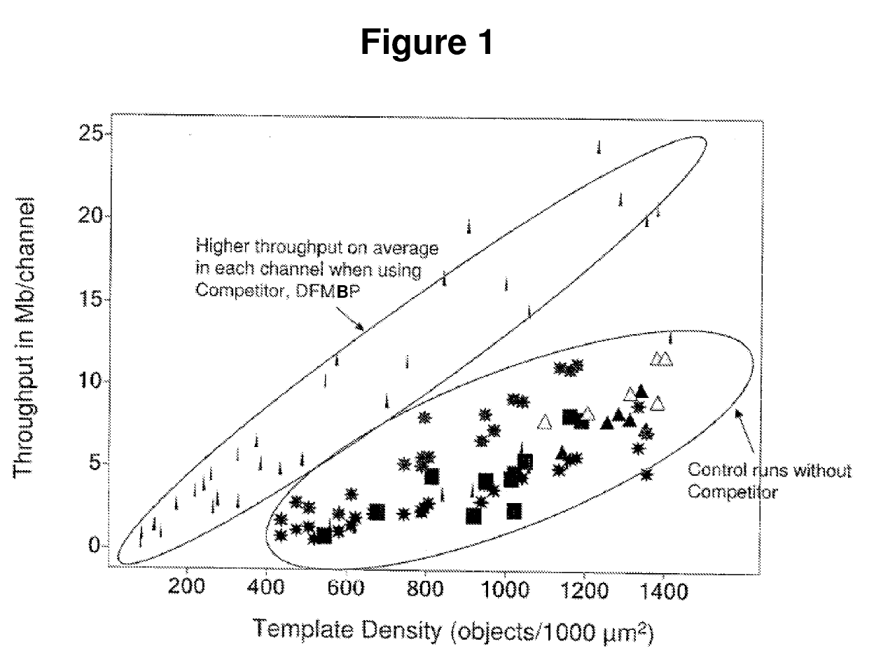

For the illustration of the sequencing process, see, e.g., U.S. patent application Ser. No. 12/043,033 (Xie et al. filed Mar. 5, 2008) and Ser. No. 12/113,501 (Xie et al. filed May 1, 2008) (e.g., FIGS. 1A and 1B). For sequencing, the slide is assembled into a 25 channel flow cell using a 50-μm thick gasket. The flow cell is placed into a Heliscope™ Sample Loader (Helicos BioSciences Corporation). A passive vacuum is built into the apparatus and is used to pull fluid across the flow cell. The flow cell is then rinsed with 150 mM HEPES/150 mM NaCl, pH 7.0 (“HEPES/NaCl”) and equilibrated to a temperature of 50° C. Separately, the nucleic acid to be sequenced is sheared to approximately 200-500 bases (Covaris), polyA tailed (50-70 average number dA's) using dATP and terminal transferase (NEB), 3′-end labeled with ATTO 647N-SS-ddUTP, and then diluted in 3×SSC to a final concentration of approximately 200 pM. A 100-μL aliquot is placed in one or more channels of the flow cell and incubated on the slide for 15 minutes. After incubation, the temperature of the flow cell is then reduced to 37° C. and the flow cell is rinsed with 1×SSC/150 mM HEPES/0.1% SDS, pH 7.0 (“SSC/HEPES/SDS”) followed by HEPES/NaCl. The resulting slide contains the primer template duplex randomly bound to the glass surface. Since the polyA/oligoT sequences are able to slide, the primer templates are filled and locked by firstly incubating the surface with Klenow exo+, TTP, in reaction buffer (NEB), washing thoroughly with HEPES/NaCl, and then incubating with Klenow exo+, dATP/dCTP/dGTP, in reaction buffer (NEB). A single step fill and lock can be done by incubating a mixture of TTP and 3 reversible terminator analogs of C, G, and A, see Virtual Terminator™ citations below. Since the Virtual Terminator™ analogs carry a dye molecule, it is possible to omit the dye label on the ddUTP used above. The slide is washed thoroughly again using the HEPES/NaCl to remove all traces of the dNTPs before initiating the actual sequencing by synthesis process. The temperature of the flow cell is maintained at 37° C. for sequencing and the objective is brought into contact with the flow cell.

Further, Virtual Terminator™ nucleotide analogs of 2′-deoxycytosine triphosphate, 2′-deoxyguanidine triphosphate, 2′-deoxyadenine triphosphate, and 2′deoxyuracil triphosphate, each having a cleavable ATTO 647N label (at the 7-deaza position for ATP and GTP and at the C5 position for CTP and UTP, see, e.g., U.S. patent application Ser. No. 12/244,698 (Siddiqi et al. filed Oct. 1, 2008), Ser. No. 12/098,196 (Efcavitch et al. filed Apr. 4, 2008), Ser. No. 11/803,339 (Siddiqi et al. filed May 14, 2007), and Ser. No. 11/603,945 (Siddiqi et al. filed Nov. 22, 2006) are stored separately in the buffer containing 20 mM Tris-HCl, pH 8.8, 75 μM MnSO₄, 10 mM (NH₄)₂SO₄, 10 mM KCl, 10 mM NaCl and 0.1% Triton X-100, and 50 U/mL Klenow exo− polymerase (NEB). In a preferred example 10-200 μM competitor is included in this solution. In a preferred example the competitor is difluoromethylene bisphosphonate (DFMBP).

Sequencing proceeds as follows. The flow cell is placed on a movable stage that is part of a high-efficiency fluorescence imaging system Heliscope™ Single Molecule Sequencer (Helicos BioSciences Corporation). First, initial imaging is used to determine the positions of duplex on the epoxide surface. The ATTO 647N label attached to the nucleic acid template fragments is imaged by excitation using a laser tuned to 635 nm radiation in order to establish duplex position. For each slide only single fluorescent molecules that are imaged in this step are counted. Next, the ATTO 647N label is cleaved off incorporated template by introduction into the flow cell of 50 mM TCEP/250 mM Tris, pH 7.6/100 mM NaCl/TCEP solution”) for 5 minutes, after which the flow cell is rinsed with SSC/HEPES/SDS and HEPES/NaCl. The template is capped with 50 mM iodoacetamide/100 mM Tris, pH 9.0/100 mM NaCl (“Iodoacetamide solution”) for 5 minutes followed by rinse with SSC/HEPES/SDS and HEPES/NaCl. Imaging of incorporated nucleotides as described below is accomplished by excitation of an ATTO 647N dye using a 635-nm radiation laser. 100 nM ATTO 647N-dCTP is placed into the flow cell and exposed to the slide for 2 minutes. After incubation, the slide is rinsed in SSC/HEPES/SDS, followed by HEPES/NaCl. An oxygen scavenger containing 30% acetonitrile and scavenger buffer (100 mM HEPES, 67 mM NaCl, 25 mM MES, 12 mM Trolox, 5 mM DABCO, 80 mM glucose, 5 mM NaI, and 0.1 U/μL glucose oxidase (USB), pH 7.0) is next added. The slide is then imaged (100-1000 frames) for 50-100 milliseconds at 635nm. The positions having detectable fluorescence are recorded. After imaging, the flow cell is rinsed with SSC/HEPES/SDS and HEPES/NaCl. Next, the ATTO 647N label is cleaved off incorporated dCTP by introduction into the flow cell of TCEP solution for 5 minutes, after which the flow cell is rinsed with SSC/HEPES/SDS and HEPES/NaCl. The remaining nucleotide is capped with iodoacetamide solution for 5 minutes followed by rinse with SSC/HEPES/SDS and HEPES/NaCl. Optionally, the scavenger is applied again in the manner described above, and the slide is again imaged to determine the effectiveness of the cleave/cap steps and to identify nonincorporated fluorescent objects.

The procedure described above is then conducted with 100 nM ATTO 647N-dATP, followed by 100 nM ATTO 647N-dGTP, and finally 100 nM ATTO 647N-dUTP. Uridine may be used instead of Thymidine due to the fact that the ATTO 647N label is incorporated at the position normally occupied by the methyl group in Thymidine triphosphate, thus turning the dTTP into dUTP. The procedure (expose to nucleotide, polymerase, rinse, scavenger, image, rinse, cleave, rinse, cap, rinse, scavenger, final image) is repeated for a total of about 80-120 cycles.

Once the desired number of cycles is completed, the image stack data (e.g., the single-molecule sequences obtained from the various surface-bound duplexes) are aligned to produce the individual sequence reads, see, e.g., U.S. patent application Ser. No. 12/187,892 (Emhoff et al. filed Aug. 7, 2008). The individual single molecule sequence read lengths obtained range from 2 to 50+ consecutive nucleotides. Only the individual single molecule sequence read lengths above some predetermined cut-off depending upon the nature of the sample, e.g. greater than 20 bases and above, are analyzed by comparing to a reference sequence.

The illustrative claims appended hereto are intended to form part of the specification as though fully reproduced therein.

1. A method comprising exposing a functional substrate to (i) a biological mixture including at least one optically detectable moiety, and (ii) a competitor effective to reduce non-specific adsorption of the detectable moiety to the functional substrate.

2. The method of claim 1, wherein the substrate is planar glass or silica.

3. The method of claim 1, wherein the substrate has defined reaction sites.

4. The method of claim 3, wherein the reaction sites are wells, vessels or microfabricated.

5. The method of claim 1, wherein the functional substrate comprises any of a primer, template, or polymerase.

6. The method of claim 1, wherein the biological mixture comprises a polymerase and nucleotide or analog thereof.

7. The method of claim 1, where the optically detectable moiety is a dye labeled nucleotide or analog thereof.

8. The method of claim 7, where the dye is a fluorophore.

9. The method of claim 8 wherein the fluorophore is negatively charged.

10. The method of claim 7, wherein the fluorophore comprises a cyanine, rhodamine, or ATTO.

11. The method of claim 1, wherein detectable moiety is an optically resolvable single molecule.

12. The method of claim 1, where the competitor is incubated with the substrate during preparation of the functional support.

13. The method of claim 1, wherein the competitor is incubated together with the optically detectable moiety.

14. The method of claim 1, wherein the competitor is a polyphosphate or polyphosphonate.

15. The method of claim 1, wherein the competitor comprises

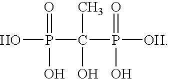

16. The method of claim 1, wherein the competitor comprises

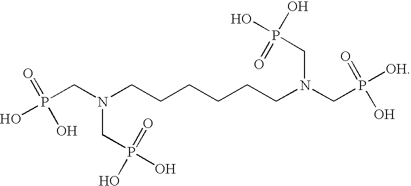

17. The method of claim 1, wherein the competitor comprises

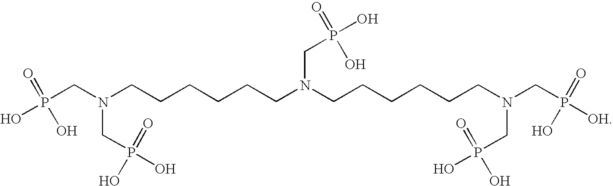

18. The method of claim 1, wherein the competitor comprises

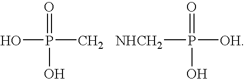

19. The method of claim 1, wherein the competitor comprises

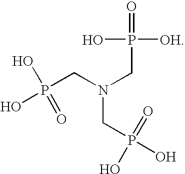

20. The method of claim 1, wherein the competitor comprises

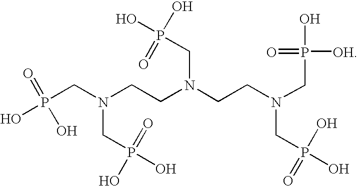

21. The method of claim 1, wherein the competitor comprises

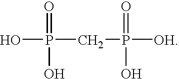

22. The method of claim 21, wherein the —CH₂— is replaced with —CF₂—.

23. The method of claim 1, wherein the competitor comprises an organic solvent.

24. The method of claim 23, wherein the solvent comprises methanol, ethanol, or acetonitrile.

25. The method of claim 1, wherein the competitor comprises at least one CF₂ and one or more phosphates or phosphonates.
